(19) United States
(12) Patent Application Publication
(10) Pub. No.: US 2005/0079604 A1
(43) Pub. Date: Apr. 14, 2005

(54) Method for improving functionality of tissue constructs

(76) Inventors: Lucie Germain, Saint-Augustin (CA); Francois A. Auger, Siliery (CA); Guillaume Grenier, Quebec (CA); Danielle Larouche, Quebec (CA); Murielle Remy-Zolghadri, Le Bouscat (FR); Francois Bergeron, Sainte-Foy (CA)

Correspondence Address: OGILVY RENAULT, 1981 MCGILL COLLEGE AVENUE, SUITE 1600, MONTREAL, QC, H3A2Y3, CA

(21) Appl. No.: 10/495,748

(22) PCT Filed: Nov. 27, 2002
(86) PCT No.: PCT/CA02/01812

Related U.S. Application Data
(60) Provisional application No. 60/333,484, filed on Nov. 28, 2001.

(51) Int. Cl. C12N 5/00; C12N 5/08

(52) U.S. Cl. 435/325; 435/366

(57) ABSTRACT

The present invention relates two new methods of improving the functionality of human or animal tissues by physically or biochemically treating the tissue prior to submitting it to different tests or before grafting it to a recipientpatient. Such a treated tissue is therefore rendered having a greater capability to resist to mechanical stress or shows a higher contractility.

### TECHNICAL FIELD

This invention is in the field of tissue engineering. It relates to methods of improvement of the functionality of various engineered tissue constructs. According to the invention, the improvement of the function of tissue constructs can be obtained by the alignment of the cells and elements of the extracellular matrix. Cells that respond in particular to alignment are smooth muscle cells and fibroblasts (or mesenchymal cells). Improvement of the functionality according to the invention is also achieved by allowing the cells, present in those tissue constructs, to reach higher levels of differentiation by modulating the composition of the cell culture medium.

### BACKGROUND OF THE INVENTION

One of the primary objectives of tissue engineering is to use cultured human cells to recreate functional tissues and organs in order to provide “replacement parts” that can be grafted into humans. Tissue engineering offers a wide variety of methods for organ reconstruction including tissue in-growth, seeding of cells on artificial or biodegradable scaffold, and collagen gels. Among them, a new method of tissue engineering has emerged that uses the method of coaxing the cells into secretion of their own extracellular matrix thus forming a living sheet. This method, called the self-assembly approach, produces sheets of living tissue of high quality that are completely devoid of exogenous scaffolds.

Living cell sheets produced by self-assembly or other methods can be used as the base material for complex tri-dimensional engineered tissue constructs. The living nature of the material makes it a dynamic environment, in which cells constantly degrades and synthesize extracellular matrix proteins. This allows for the fusion of living sheets that are pressed together in a particular shape and with appropriate mechanical support to fuse and make thus creating a whole tissue after a certain amount of time. As examples, living sheets can either be stacked on each other in order to create thick multi-layered tissue constructs or rolled on a cylinder to create tubular structures. These methods have been used with great success in the reconstruction of tissues like human blood vessel, skin and cornea.

The strength of and the mechanical properties of living tissues, either natural or engineered, lies in the fibers of extracellular matrix that are synthesized by the cells inside the tissue. But these fibers do not need only to be synthesized, they also need to be properly oriented in their tri-dimensional environment and to be anchored to the rest of the fibrous network. Until now the development of tissue engineering methods to produce reconstructed tissues has focused on the optimization of histological properties of the tissues. However, the functional aspects of the reconstructed tissues that are related to global fiber and cell alignment, such as mechanical strength and contractile response, should also be as close as possible to the functionality of the native tissues. This approach has been recently described as the functional tissue-engineering. For example, it is known that the self-assembly approach can be used to produce a reconstructed human vascular media (RHVM) for pharmacological studies from cultured human vascular smooth muscle cells. This construct was shown as pioneer for an in vitro human contractile RHVM and it displays many of the functional characteristics of normal human vessel from which the cells were originally isolated. Nevertheless the contractile/relaxation levels of its responses were smaller than those observed for the umbilical vein from which it originates.

The control of the orientation of cells and of the extracellular matrix fibers appears to be relevant to organ functions. For example, there are many tissues in which it is known that cells, especially cells of mesenchymal origin, are oriented. In native tissues, cells and extracellular matrix fibers often present a characteristic orientation. This is true for a wide variety of tubular tissues such as bronchi, blood vessel, gastro-intestinal and urogenital tracts, and other tissues such as muscle and ligament.

Assembling tissues from living sheets is an efficient way to produce tissue-engineered constructs of these organs. However, there is a need of technologies that allows an increase in functionality of these constructs. This can be achieved by inducing the proper orientation of the cells and the matrix fibers they are surrounded with. This should lead to tissue-engineered products with more nature-like characteristics for replacement or in vitro tests.

### SUMMARY OF THE INVENTION

One object of the present invention is to provide methods to improve the functionality of sheet-based tissue-engineered constructs by inducing a desired alignment pattern of cells and extracellular matrix fibers. This is done, by giving the appropriate mechanical support to the living tissue sheet, in order to induce alignment of the cells and their extracellular matrix fibers. Aligned living sheets can be used to produce three-dimensional tissue constructs that show improved functionality. A tubular construct, for example a reconstructed human vascular media (RHVM), can be prepared according to the present method using the self-assembly approach. The RHVM made of an aligned living sheet has a greater contractile response than a RHVM made of a sheet in which cells were not aligned. A planar construct, for example a reconstructed human skin (RHS) comprising a dermis and an epidermis, can be produced with living sheets containing aligned skin fibroblasts and aligned extracellular matrix fibers.

The mechanical strength and resistance of this planar construct is improved compared to RHS made of skin fibroblast living sheets in which the cells and the extracellular matrix fibers are distributed randomly. This indicates that the alignment of skin fibroblasts and extracellular matrix fibers greatly improves the mechanical strength of a planar construct such as RHS.

It is also an object of the present invention to provide a method to increase the differentiation level of cells present in tissue constructs, by using cell proliferation inhibitors. A tubular construct, in this case a RHVM, cultured with such cell proliferation inhibitors, has an increased contractile response compared to RHVM cultured without inhibitors.

Another object of the invention is to provide a method of increasing the functionality of a human or an animal tissue comprising attaching the tissue for a period of time sufficient for causing the organization of cells and extracellular matrix components contained in the tissue.

Improving the functionality of a human or an animal tissue may comprise culturing the tissue in a medium containing a cell proliferation inhibitor or a cell cycle inhibitor for a period of time for inducing differentiation of cells contained in the tissue.

The functionality may be at least one of mechanical resistance, contractility, or responsiveness of the cells to biologically active compounds selected from the group consisting of a biologically active agent.

The tissue used to perform the method of the invention may be a biopsy or a tissue construct obtained by in vitro culture of cells assembled in a self-produced matrix. The tissue also can be a tubular or a planar construct, a vascular tissue, a skin tissue, a corneal tissue, a valve tissue, a connective tissue or a mesenchymal tissue.

The organization of the cells in the tissue can be a parallel, transversal, or linear alignment of the cells.

Another object of the present invention is the use of cells that are generally mesenchymal or mesodermic cells.

According to another object of the invention the cell type of the invention may be selected from the group consisting of smooth muscle cell, fibroblast, skeletal muscle cell, endothelial cell, epithelial cells, nervous cell, ectodermic cell types, and adult or embryonic stem cells, or a combination thereof.

Also, the cells can be genetically altered cells or contain a genome genetically altered by mutation, deletion, or insertion.

The cell proliferation inhibitor or cell cycle inhibitor of the present invention may be the heparin or olomoucine.

### BRIEF DESCRIPTION OF THE DRAWINGS

FIG. 1. illustrates the method to align smooth muscle cells and extracellular matrix fibers in a living sheet for the elaboration of a tubular construct, in this case a reconstructed human vascular media;

FIG. 2. illustrates a macroscopic aspect of a living sheet containing smooth muscle cells attached at the opposite edges of a plastic frame at day 0 and at day 7;

FIGS. 3A to 3E illustrate a microscopic aspect of an aligned living sheet as a function of maturation time between day 0 and day 7;

FIG. 4. shows confocal images of immunolabeled smooth muscle α-actin and collagen I, proteins of cell cytoskeleton and extracellular matrix respectively, in a living sheet at day 0 and day 7, according to the method of the invention;

FIG. 5. illustrates a dose-response curve showing the contraction of a tubular construct in response to cumulative doses of histamine, in this case a reconstructed human vascular media prepared (RHVM) with non aligned or aligned living sheets;

FIG. 6. illustrates the microscopic aspect of reconstructed human vascular media (RHVM) prepared with living sheet containing non-aligned or aligned smooth muscle cells and their extracellular matrix, and stained with Masson's trichrome;

FIGS. 7A to 7F illustrate the method used to align the living sheets necessary for the preparation of a planar structure, in this case a reconstructed human skin (RHS);

FIG. 8. illustrates the resistance of a planar construct. Graphic shows representative curves of ultimate strength as a function of stretch distance of reconstructed human skin (RHS) prepared with living sheets containing non-aligned or aligned fibroblasts and their extracellular matrix;

FIG. 9. illustrates a dose response curve showing the contraction in response to cumulative doses of histamine of a tubular construct, in this case reconstructed human vascular media (RHVM) which were cultured in the presence or absence of cell proliferation inhibitors; and

FIG. 10. shows microscopic images of immunolabeled differentiation markers of smooth muscle cells present in reconstructed human vascular media, which were cultured in the presence or absence of cell proliferation inhibitors.

### DETAILED DESCRIPTION OF THE PREFERRED EMBODIMENT

Cells of mesenchymal origin (such as smooth muscle cells and fibroblasts) are grown as a multilayer of cells intertwined in a complex and physiological extracellular matrix synthesized by the cells themselves. When these cells are maintained in culture several days post confluence, cells and matrix detach or can be detached as a whole from the culture substratum, thus creating a living sheet of cells in a complex and physiological fibrous matrix of endogenous origin. This living sheet can then be cultured while the sheet length is kept constant. In accordance with the present invention the living sheet can be further mounted and attached at both ends of a plastic frame. With time, a tension develops, and the loosely attached living sheet tightens as the cells pull on the collagen fibers during the compaction of the tissue. As a consequence, the cells and extracellular matrix align along the axis of the tension. When the living sheet shows alignment of the cells and extracellular matrix fibers, it is used for the reconstruction of three-dimensional tissue constructs with improved functionality.

Culture conditions also influence the functionality of the tissue produced. In accordance with the present invention, the functionality of the tissue constructs can be improved by increasing the differentiation level of the cells present in the tissue construct by adding cell proliferation inhibitors to the culture media. After the treatment, the cells have reached a higher level of differentiation compared to cells present in tissue constructs cultured in absence of cell proliferation inhibitors.

In another embodiment of the present invention, a tissue can be constructed from living sheets in which the cells have been aligned transversally or longitudinally by mechanical restraints in order to improve its physiological, biochemical or metabolic functions.

In another embodiment of the present invention, a tissue can be constructed from living cells and a scaffold in which the cells have been seeded.

The following examples describe methods for improving the functionality of engineered tissue constructs. Two tissue constructs, a contractile tubular construct (RHVM) as well as a planar tissue construct (RHS) are used to demonstrate the effect of these methods on the functionality of these tissues. These examples are given to illustrate the invention rather than to limit its scope.

### EXAMPLE I

### Improved Functionality of Reconstructed Human Vascular Media (RHVM) Prepared with a Living Sheet Containing Aligned Smooth Muscle Cells and Extracellular Matrix Fibers

Viable sub-cultured human smooth muscle cells (passages 3-7) were seeded at a density of 10 000 cells/cm² in a standard 75 cm² culture flask. Cells were fed with 15 ml of culture medium containing Dulbecco's Modification of Eagle's Medium™ and Ham's F12 Modified Medium™ (3:1 mixture), 10% Fetal Clone II (Hyclone™), 100 U/ml of penicillin G and 25 μg/ml of gentamicin. The culture medium was changed three times per week. A freshly prepared solution of ascorbic acid was added each time the medium was changed at a final concentration of 50 μg/ml. Cells were kept in a humidified atmosphere (92% air and 8% CO₂).

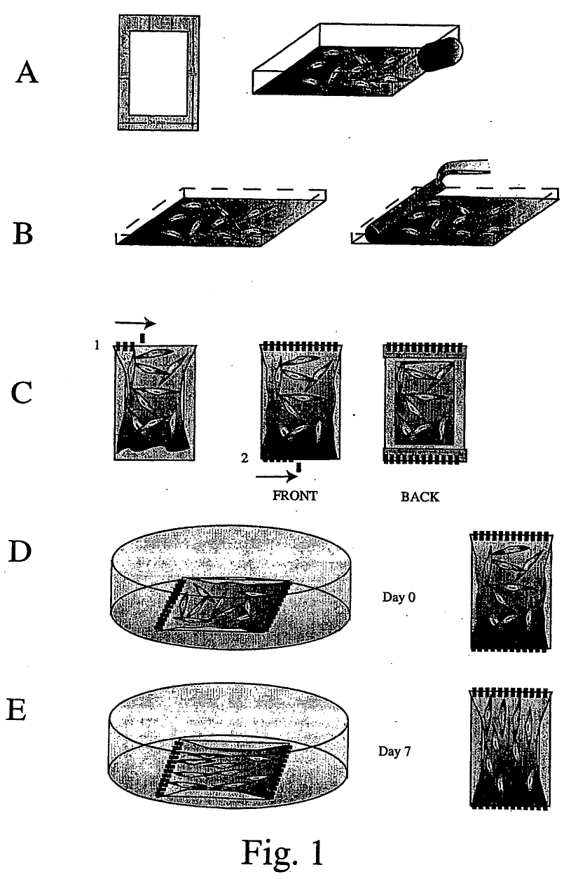

Under the above-mentioned culture conditions, the cells will adhere to the plastic culture flask and will proliferate until the entire culture surface is covered with cells (confluence). If the culture conditions are maintained, the cells will synthesize fibrous material. If the culture is prolonged for several additional days, this fibrous tissue will show signs of detachment from the culture substratum and will spontaneously completely detach itself, as a whole, from the substratum. It is also possible to induce the detachment of the forming sheet, for example in order to control the time of maturation. One possibility is to open the flask (FIG. 1B part I) and to use a rubber policeman or fine tweezer to carefully detach the sheet from the culture surface (FIG. 1B part II) when signs of detachment are apparent.

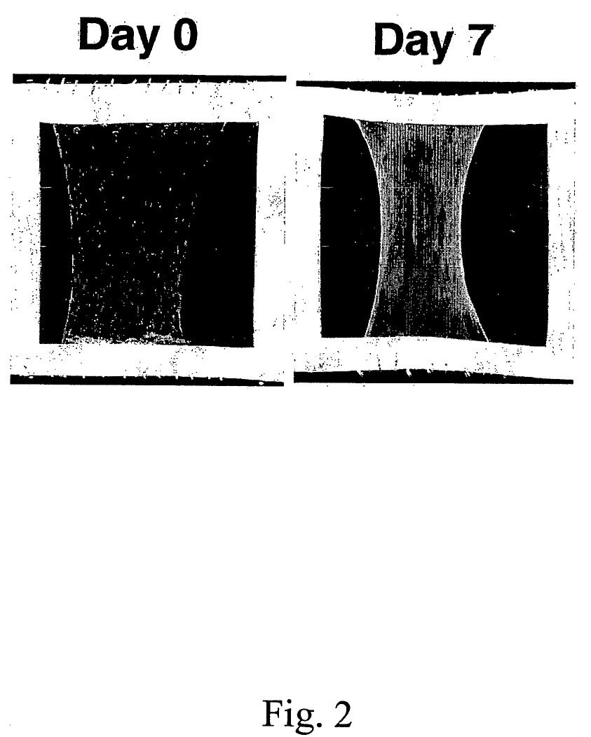

The method used to align the living sheets of smooth muscle cells for the elaboration of the tubular construct is illustrated in FIG. 1. Once the living sheet was detached from the culture surface, the extremity of the detached living sheet was rapidly, but carefully, attached on one side of the plastic frame by gently clipping it using Ligaclip™ (FIG. 1C part I). The other sheet extremity is then clipped on the opposite side of the plastic frame (FIG. 1C part II). Then, the plastic frame, on which the sheet was clipped, was deposited in a bacteriological petri dish containing culture medium supplemented with ascorbic acid (FIG. 1D part I). At this point, the attached living sheet was loose and the cells present in the living sheet were randomly oriented (FIG. 1D part II; FIG. 2). After 7 days of culture, the living sheet became tighter and oriented along the axis of the tension that was generated by the cells pulling on the collagen fibers (FIG. 2).

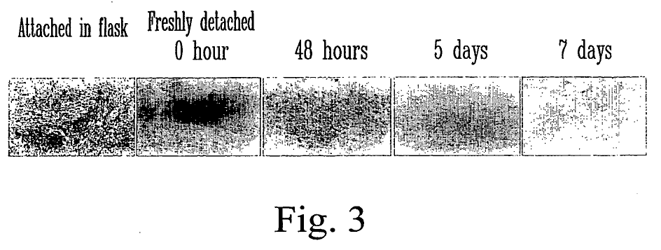

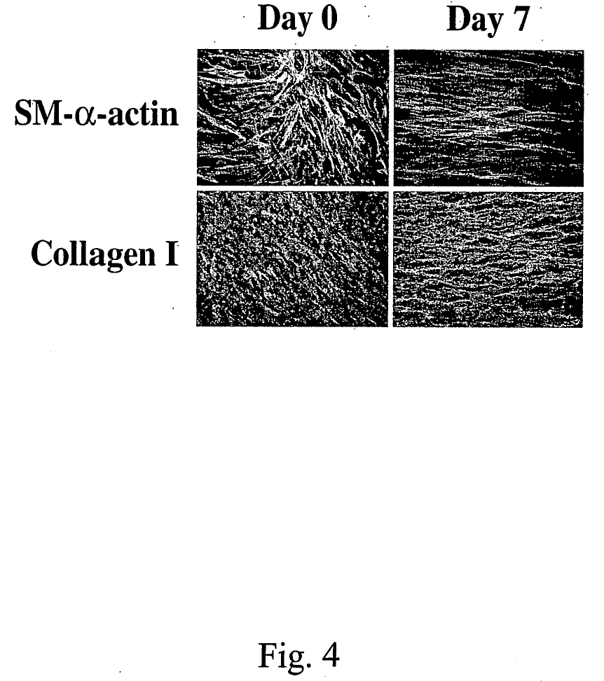

FIG. 3 shows a microscopic view of the living sheet as a function of maturation time. The cells attached on the culture surface were randomly oriented (FIG. 3A). Once detached from the culture surface (0 hour; FIG. 3B), the latter structure spontaneously contracted and appeared as a dark zone constituted of clustered cells. After 48 hours, this zone tended to decluster as cells contracted in a uniaxial direction (FIG. 3C). Finally, cells continued to reorganize along the strain with time (FIG. 3D) until a parallel orientation of cells and extracellular matrix fibers was visible after 7 days (FIGS. 3E and 4) as shown by the alignment of smooth muscle alpha-actin and collagen I. These two proteins are related to cell cytoskeleton and extracellular matrix, respectively.

In order to give a cylindrical form to the aligned living sheet, the sheet was rolled on a tubular support. One edge (one of the two edges that were attached) of the aligned living sheet is placed between the tubular support and a thread. The thread was then pulled along the arrow in order to squeeze one edge of the sheet between the thread and the external surface of the tubular support. At this instance, a minimal amount of the sheet should cross over the thread although it was important that all the edges be secured. While rolling the living sheet, a sustained tension force has to be applied in order to prevent the retraction of the living sheet. When the sheet was completely rolled up, the thread was slid off. The sheet is then again secured with the thread to prevent unrolling of the sheet. The thread may be removed 1-2 days later. The tubular living tissue can be cultured for several weeks, with ascorbic acid, to allow further maturation of the tissue. Three-dimensional vascular constructs were fabricated using living sheets containing cells and extracellular matrix that had been aligned or not beforehand.

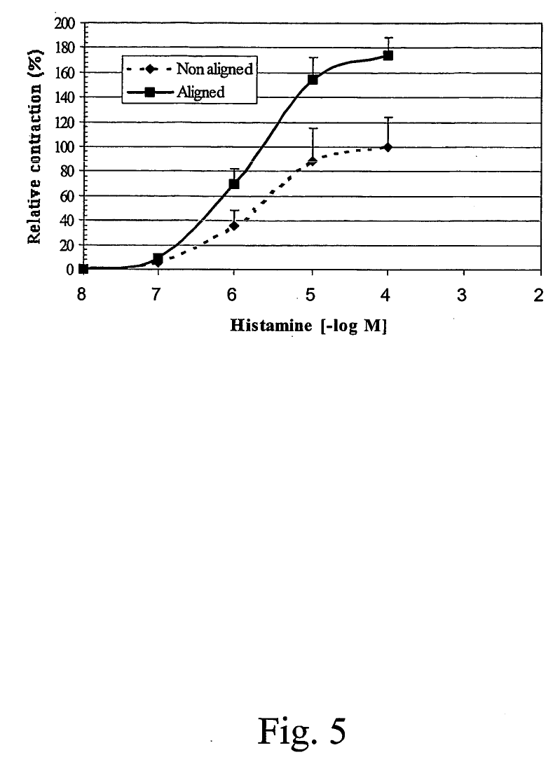

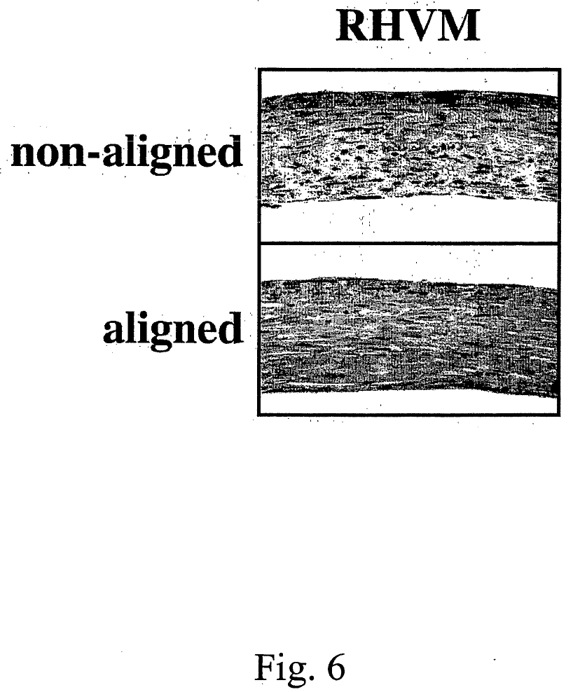

In order to evaluate the functionality of the tubular tissue constructs, the reconstructed human vascular media (RHVM), was slid off its tubular support and cut into annular sections of 2 to 5 mm. These annular sections were used to test the contraction of the RHVM in vitro. The annular sections prepared according to the present invention were tested with histamine, a physiological vasoactive substance. Isometric tension generated by the RHVM contraction was directly recorded via a force transducer (Kilster-Morse, DSG BE4). FIG. 5 shows the contraction response of annular sections of the tubular constructs when stimulated with cumulative doses (10⁻⁸-10⁻⁴ mol/L) of histamine. The results obtained indicate that alignment of the living sheet prior to construction of the RHVM results in an increase of the contractile response. Indeed, the contractile response induced by histamine showed by these tubular constructs was greater than that obtained contraction for an RHVM made from a living sheet that had not been aligned before rolling. This may be due in part to the final orientation of cells and extracellular matrix fibers observed in the RHVM (FIG. 6).

The tubular support used for elaboration of the constructs can be made of various materials and diameters in order to produce diverse lumens' caliber. It is also possible to roll more than one aligned living sheet in various orientations in order to obtain tissue with multidirectional layers. It is not intended to limit the scope of this invention to one particular shape or cell origin. One skilled in the art can readily appreciate that various modifications can be applied to the method without departing from the scope and spirit of the invention.

Hence, we were able to obtain contractile response of an aligned living sheet made either of smooth muscle cells or perivascular fibroblasts.

### EXAMPLE II

### Preparation of a Reconstructed Human Skin (RHS) from Living Sheets Containing Aligned Fibroblasts and Extracellular Matrix Fibers

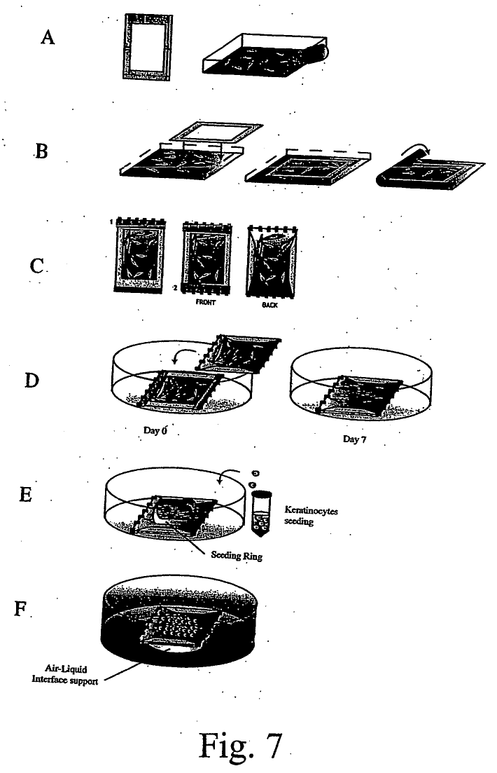

The method used to align the living sheets of fibroblasts for the elaboration of the planar construct such as a reconstructed human skin (RHS) is illustrated on FIG. 7. Dermal fibroblasts are seeded at 8000 cells/cm² in a standard 75 cm² culture flask and cultured for 35 days in fibroblast culture medium containing Dulbecco-Vogt modification of Eagle's (DME™) medium, 10% fetal calf serum (Hyclone™), 100 UI/ml penicillin G (Sigma) and 25 μg/ml gentamicin (Sigma), supplemented with 50 μg/ml of freshly prepared ascorbic acid solution until the formation of a living sheet that can be manipulated. Culture medium was changed three times a week.

In order to produce the dermal portion of the RHS, a plastic frame was deposited on a mature sheet and one of the extremity of the living sheet detached and folded down on the frame (FIG. 7B). Ligaclip™ were then used to fix both opposite extremities of the sheets on the frame (FIG. 7C). After the living sheet was peeled off from the button of the flask, two fibroblast sheets mounted on their respective plastic frame were superimposed (FIG. 7D) and a sponge is then added on the surface of the construct for one day to allow the cohesion between the sheets. Culture medium was changed three times a week.

In order to produce the epidermal portion of the RHS, 2×10⁵ human keratinocytes/cm² were seeded on the reconstructed dermis after 7 days. The RHS was then cultured in keratinocyte medium containing DME with Ham's F12 (3:1 proportion) supplemented with 10% fetal calf serum (Hyclone™), 10 ng/ml epidermal growth factor (EGF) (Austral Biologicals), 24.3 μg/ml adenine (Sigma), 5 μg/ml insulin (Sigma), 2×10⁻⁹ M 3,3′,5′triiodo-L-thyronine (Sigma), 5 μg/ml human transferrin (Roche), 0.4 μg/ml hydrocortisone (Calbiochem), 10⁻¹⁰ M cholera toxin (ICN Biochemical), 100 UI/ml penicillin G (Sigma) and 25 μg/ml gentamicin (Sigma). Culture medium was supplemented with 50 μg/ml of ascorbic acid. The keratinocytes reached confluence after 8 days of submerged culture. To improve the epidermal differentiation, the RHS clipped on the plastic frame, was raised at the air-liquid interface and cultivated with air-liquid medium, i.e. keratinocyte medium described above without EGF, and supplemented with 50 μg/ml of ascorbic acid. Culture medium was changed three times a week. After 21 days of culture, the RHS was processed for mechanical testing.

Rupturing points of freshly detached LTS (n=4) and with LTS containing non-aligned (n=4) and aligned (n=4) components were measured directly using a semi-automated mechanical stretching apparatus. Both extremities of LTS were fixed on anchoring jaws, one mobile and the other connected to a cell force transducer. The aligned LTS were stretched in the parallel direction of the orientation of its components (cells and ECM). Ultimate strength (rupturing points) of RHS prepared with freshly detached living sheet (n=4) and with living sheet containing non-aligned (n=4) and aligned (n=4) components were measured directly using a semi-automated mechanical stretching apparatus. Both extremities of a RHS rectangular strip extremities were fixed on anchoring jaws, including one mobile and one connected to a cell force. The aligned RHS were stretched in the parallel direction to the orientation of the living sheet, i.e. to cells and to extracellular matrix components. Concerning the LTS in which the components were not aligned, they were randomly attached by its opposite sides. Once anchored, the apparatus begins to stretch by pulling on the mobile jaw and the data generated from the developed constraints (resistance) as a function of the distance are recorded and processed using an acquisition software. The force (N) and the tensile stress are calculated by dividing the force by the initial cross-sectional area of the RHS. The strain is calculated by dividing the change in length of the RHS by its original length. From this constraint-deformation graphic, elasticity or stiffness (slope of the linear portion of the curve) and the ultimate tensile strength (stress at peak load) of the RHS were determined.

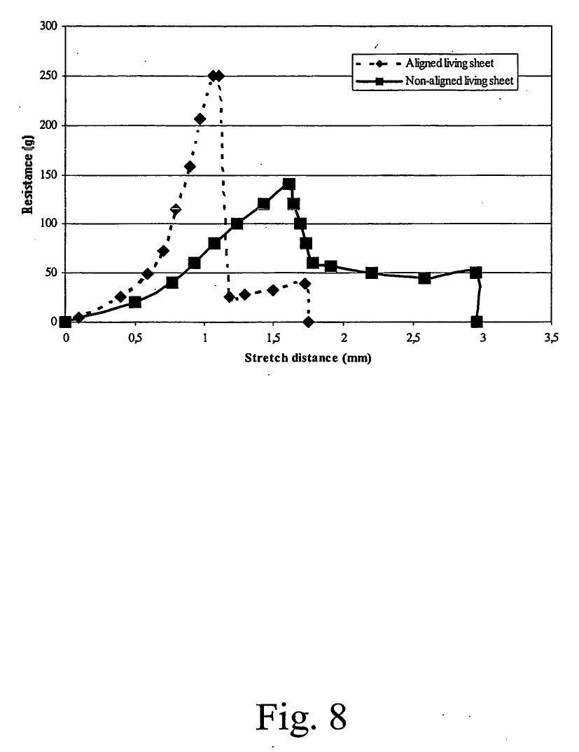

FIG. 8 shows the resistance as a function of the stretching distance of RHS using non-aligned or aligned living sheets. The rupturing point (as indicated by arrows in FIG. 8) of the RHS made of an aligned living sheet was twice as resistant when compared to RHS made of non-aligned living sheet. This result indicates that the use of an aligned living sheet increases the functionality of RHS, as measured by its resistance.

### EXAMPLE III

### Improved Functionality of a Reconstructed Human Vascular Media Constructs (RHVM) using Cell Proliferation Inhibitors

Reconstructed human vascular media (RHVM) were prepared for the control tissue construct (not aligned) as described in Example I. The RHVM used for this example were treated as follow: RHVM cultured in medium described above represent the control condition (non-treated RHVM) and RHVM supplemented with cell proliferation inhibitors, heparin or olomoucine (treated RHVM).

To assess the contractile function of RHVM which were treated or not (control condition), RHVM rings of 5-7 mm in length were removed from the tubular support used for culture after 21 days of maturation. Rings were mounted in a myograph and challenged in the presence of cumulative doses of histamine, a vasoactive agent. Isometric tension generated by RHVM contraction was directly recorded via a force transducer (Kilster-Morse, DSG BE4).

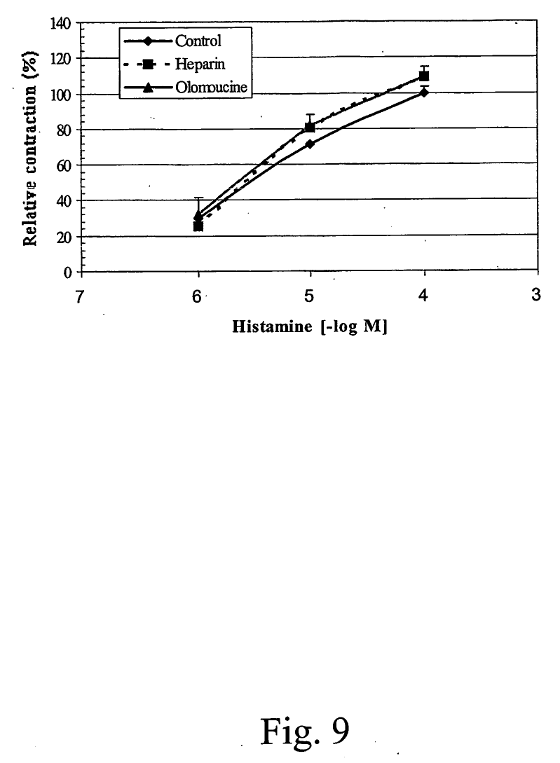

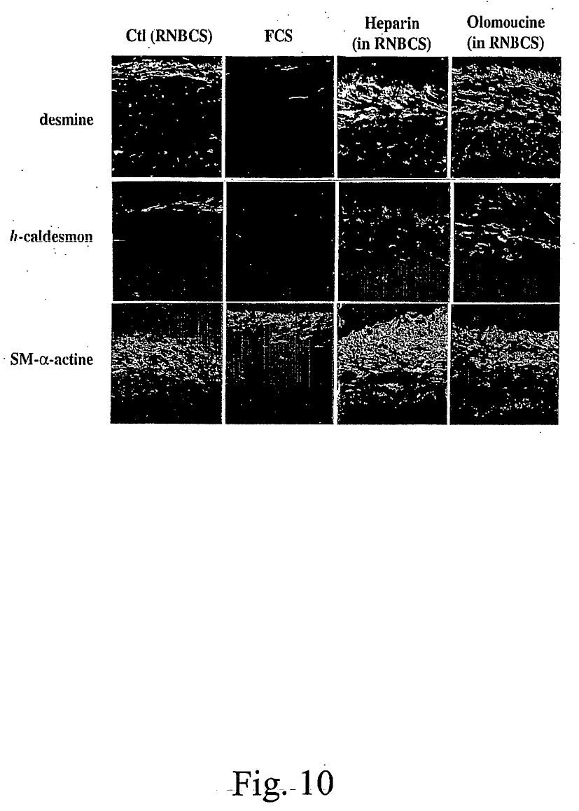

As indicated in FIG. 9, no significant difference of contraction was noted between treated and non-treated RHVM in the presence of low dose of histamine (10⁻⁶ M). RHVM cultured with heparin or olomoucine show a significantly greater contraction at higher doses of histamine, i.e. 10⁻⁵ and 10⁻⁴ M, when compared to control RHVM. These results are in accordance with FIG. 10, in which the expression of differentiation markers is increased in cells cultured in the presence of olomoucine or heparin when compared to the cells that where not cultured with the cell proliferation inhibitors.

The mechanical stability of biological tissues produced by tissue engineering represents a challenge. The skin, for example, which protects internal structures of the body, must support important mechanical stress. Furthermore, the reconstructed skin produced for grafting purpose must be resistant, stable and must have good esthetical quality. Similarly, the functionality of tubular organs, such as bronchi, blood vessel, gastro-intestinal and urogenital tracts, has been demonstrated to be dependent on the differentiation levels of cells and on the orientation of cells and extracellular matrix.

In this invention we propose two strategies allowing a enhanced functionality of the reconstructed tissues. One strategy focuses on the use of aligned living sheets for the preparation of tissue constructs and the other one on the use of cell proliferation inhibitors in order to increase the differentiation level of cells present in the tissue constructs.

The alignment of cells and extracellular matrix fibers in a living sheet for the production of a reconstructed human vascular media leads to the improvement of the contractile function of the tissue-engineered equivalent. Improvement can also be obtained by supplementation of the culture medium with cell proliferation inhibitors. Likewise, reconstructed human skin in which the dermis contained aligned fibroblasts shows excellent mechanical strength.

These reconstructed tissues could present specific advantages particularly at anatomic sites where the physical stress is high (e.g. reconstructed skin grafting on articulations). Furthermore, a reorganized extracellular matrix in reconstructed human skin dermis could greatly improve the esthetical results after grafting. Indeed, since fibroblasts contract the collagen fibers of the extracellular matrix in the direction of their orientation, aligned skin fibroblasts in skin reconstruction should allow controlling the contraction and thus improving the quality of healing. The contractile properties of the reconstructed human vascular media could be used for the replacement of coronary arteries in particular. But this reconstructed human vascular media could also be an interesting model for pharmacological studies as well as an in vitro model for fundamental research on the understanding of mechanisms of vascular physiology and physiopathology of vasculature.

While the invention has been described in connection with specific embodiments thereof, it is understood that it is intended to cover any variations, uses, or adaptations of the invention that follow, in general, the principles of the invention, including such departures from the present disclosure, as come within known or customary practice within the art to which the invention pertains and as may be applied to the essential features herein before set forth, and as follows in the scope of the appended claims.

1. A method for increasing the functionality of a human or an animal tissue by applying at least one mechanical strain in at least one orientation to said tissue or components of said tissue for a period of time sufficient for causing the organization of cells and extracellular matrix components contained in said tissue or in components of said tissue.

2. The method of claim 1, wherein said tissue comprises at least one sheet of living tissue to form living tissue sheets.

3. The method of claim 2, wherein said living tissue sheets are assembled into tissue constructs.

4. A method of claim 1 comprising culturing said tissue in a medium containing a cell proliferation inhibitor or a cell cycle inhibitor for a period of time sufficient for inducing differentiation of cells contained in said tissue.

5. The method of claim 4, wherein said cells when differentiated are cultures for a period of time sufficient for the assembly of said cells into a tissue construct.

6. The method of claim 1, wherein said functionality is at least one of the following: mechanical resistance, contractility, transparency or responsiveness of the cells to biologically active compounds.

7. A method of claim 1, wherein said functionality could be improved by the cyclic traction, the pulsatile pressure, or a combination thereof.

8. The method of claim 1, wherein said tissue is a biopsy.

9. The method of claim 1, wherein said tissue is a tissue construct obtained by in vitro culture of cells assembled in a self-produced matrix.

10. The method of claim 1, wherein said tissue is a tissue construct obtained by in vitro culture of cells seeded onto a scaffold.

11. The method of claim 1, wherein said tissue is tubular or planar.

12. The method of claim 1, wherein said tissue is a vascular tissue, a skin tissue, a corneal tissue, a valve tissue, a connective tissue or a mesenchymal tissue.

13. The method of claim 1, wherein said organization is a parallel, transversal, or linear alignment of said cells, or a combination thereof

14. The method of claim 1, wherein said cells are mesenchymal cells or mesodermic cells.

15. The method of claim 1, wherein said cells are selected from the group consisting of smooth muscle cells, fibroblasts, skeletal muscle cells, endothelial cells, nervous cells, ectodermic cells, adult or embryonic stem cells, and a combination thereof.

16. The method of claim 1, wherein said cells are genetically altered cells.

17. The method of claim 15, wherein said cells are genetically altered by mutation, deletion, or insertion.

18. The method of claim 3, wherein said cell proliferation inhibitor or cell cycle inhibitor is heparin or olomoucine.
